(19) United States
(12) Patent Application Publication
(10) Pub. No.: US 2025/0127485 A1
(43) Pub. Date: Apr. 24, 2025

(54) SYSTEM AND METHOD FOR AUTOMATED OPTIC NERVE SHEATH MEASUREMENT BASED ON THREE-DIMENSIONAL ULTRASOUND DATA

(71) Applicant: GE Precision Healthcare LLC, Waukesha, WI (US)

(72) Inventors: Aaron DENTINGER, Latham, NY (US); David MILLS, Niskayuna, NY (US); Menachem HALMANN, Monona, WI (US); Alexander SOKULIN, Kiryat Tivon (IL); Radhika MADHAVAN, Latham, NY (US)

(21) Appl. No.: 18/493,388

(22) Filed: Oct. 24, 2023

(51) Int. Cl. A61B 8/10; A61B 8/00; A61B 8/08; G06T 7/00; G06T 7/162

(52) U.S. Cl. CPC A61B 8/10; A61B 8/4245; A61B 8/466; A61B 8/5207; A61B 8/523; G06T 7/0012; G06T 7/162

(57) ABSTRACT

An ultrasound imaging system and method for determining a measurement of an optic nerve sheath are provided. The ultrasound imaging system and method may include controlling an ultrasound probe to acquire three-dimensional (3D) ultrasound data of the optic nerve sheath. The ultrasound imaging system and method may include determining the measurement of the optic nerve sheath based on the 3D ultrasound data. The ultrasound imaging system and method may include controlling the display to display information related to the measurement of the optic nerve sheath.

### BACKGROUND

A person may experience increased intracranial pressure (ICP) as a result of a primary injury, such as head trauma, an underlying health problem, or the like. Further, increased ICP may aggravate secondary injuries, such as ischemia, cerebral edema, hypoxia, or the like, that manifest after the primary injury. The optic nerve sheath may increase in size as a result of increased ICP. Accordingly, an enlargement of the optic nerve sheath may be indicative of elevated ICP.

### SUMMARY

This summary introduces concepts that are described in more detail in the detailed description. It should not be used to identify essential features of the claimed subject matter, nor to limit the scope of the claimed subject matter.

In an aspect, an ultrasound imaging system may include an ultrasound probe, a display, and one or more processors configured to control the ultrasound probe to acquire three-dimensional (3D) ultrasound data of an optic nerve sheath, determine a measurement of the optic nerve sheath based on the 3D ultrasound data, and control the display to display information related to the measurement of the optic nerve sheath.

In another aspect, a method may include controlling an ultrasound probe to acquire three-dimensional (3D) ultrasound data of an optic nerve sheath, determining a measurement of the optic nerve sheath based on the 3D ultrasound data, and controlling the display to display information related to the measurement of the optic nerve sheath.

In another aspect, an ultrasound imaging system may include a memory configured to store instructions, and one or more processors configured to execute the instructions to control an ultrasound probe to acquire three-dimensional (3D) ultrasound data of an optic nerve sheath, determine a measurement of the optic nerve sheath based on the 3D ultrasound data, and control a display to display information related to the measurement of the optic nerve sheath.

Some embodiments of the present disclosure provide an improved ultrasound imaging system that improves the accuracy, efficiency, speed, and reliability of ultrasound data acquisition and optic nerve sheath measurement, provide an improvement to the technical field of ultrasound imaging, and provide an improvement to patient safety and treatment.

### BRIEF DESCRIPTION OF DRAWINGS

FIG. 1 is a diagram of an ultrasound imaging system according to an embodiment.

FIG. 2 is a flowchart of an example process for determining a measurement of an optic nerve sheath according to an embodiment.

FIG. 3 is a diagram of displaying preview ultrasound images in real-time according to an embodiment.

FIG. 4 is a flowchart of an example process for generating scan guidance information according to an embodiment.

FIG. 5 is a diagram of displaying scan guidance information according to an embodiment.

FIG. 6 is a flowchart of an example process for controlling an ultrasound probe to automatically acquire 3D ultrasound data based on a scan quality metric according to an embodiment.

FIG. 7 is a flowchart of an example process for determining a measurement of the optic nerve sheath based on 3D ultrasound data according to an embodiment.

FIG. 8 is a diagram of long-axis measurement planes according to an embodiment.

FIG. 9 is a diagram of a short-axis measurement plane according to an embodiment.

FIG. 10 is a diagram of measurements of the optic nerve sheath according to an embodiment.

FIG. 11 is a flowchart of an example process for determining a measurement of the optic nerve sheath at a measurement position at a predetermined distance from an anatomical landmark based on 3D ultrasound data according to an embodiment.

FIGS. 12A-12C are diagrams of ultrasound images for determining a measurement of the optic nerve sheath based on 3D ultrasound data according to an embodiment.

FIG. 13 is a flowchart of an example process for determining a measurement of the optic nerve sheath using a plurality of measurements based on 3D ultrasound data according to an embodiment.

FIGS. 14A and 14B are diagrams of ultrasound images for determining a measurement of the optic nerve sheath based on 3D ultrasound data according to an embodiment.

FIG. 15 is a flowchart of an example process for determining a measurement of the optic nerve sheath using an AI model inference based on 3D ultrasound data according to an embodiment.

FIG. 16 is a diagram of a display of information related to a measurement of the optic nerve sheath according to an embodiment.

FIG. 17 is a diagram of an example process for training an AI model inference according to an embodiment.

### DETAILED DESCRIPTION

Embodiments of the present disclosure will now be described, by way of example, with reference to the figures.

Ultrasound imaging systems and methods may use two-dimensional (2D) ultrasound imaging to aid medical personnel in determining a measurement of the optic nerve sheath. In this way, the ultrasound imaging systems and methods that utilize 2D ultrasound data might provide relatively rapid and non-invasive assessment of ICP through measurement of the optic nerve sheath. However, the ultrasound imaging systems and methods might involve optimal alignment of the ultrasound probe with respect to an anatomical plane of the optic nerve sheath in order to acquire a 2D ultrasound image that accurately reflects a true measurement of the optic nerve sheath.

Accordingly, ultrasound imaging systems and methods that utilize 2D ultrasound data might involve a skilled operator spending sufficient time and imaging resources to position the ultrasound probe while acquiring the 2D ultrasound data. Thus, ultrasound imaging systems and methods that utilize 2D ultrasound data might not be optimally efficient for being implemented at a point of care, a point of injury or in a medical vehicle during transport to a treatment location. Further, ultrasound imaging systems and methods that utilize 2D data might involve a skilled reviewer assessing 2D ultrasound images to determine a measurement of the optic nerve sheath. If the ultrasound probe was misaligned with respect to the optic nerve sheath during image acquisition, then the 2D ultrasonic images might result in optic nerve sheath measurements that have suboptimal accuracy, reliability, etc. Also, if the anatomy of the optic nerve sheath is tilted, curved, etc., then the 2D ultrasound images might not accurately reflect the true size of the optic nerve sheath, and might result in inaccurate optic nerve sheath measurements.

The ability of medical personnel to accurately identify elevated ICP and routinely monitor changes in ICP is valuable. Further, the ability to quickly and efficiently identify elevated ICP in a resource-limited setting might be valuable. For instance, medical personnel might desire to determine ICP at a point of care, a point of an injury or during transport in a medical vehicle in order to accurately determine an appropriate treatment location. As another example, medical personnel might desire to identify elevated ICP in a large number of casualties at a site of mass-casualty event in order to triage the victims.

Some embodiments of the present disclosure provide an improved ultrasound imaging system that improves the accuracy, efficiency, speed, and reliability of ultrasound data acquisition and optic nerve sheath measurement, provide an improvement to the technical field of ultrasound imaging, and provide an improvement to patient safety and treatment. For instance, some embodiments of the present disclosure provide an ultrasound imaging system that may acquire 3D ultrasound data of an optic nerve sheath, and determine a measurement of the optic nerve sheath using the 3D ultrasound data. Further, some embodiments of the present disclosure provide an ultrasound imaging system that may utilize artificial intelligence (AI) models for scan guidance, segmentation, measurement, and/or diagnosis. In this way, some embodiments of the present disclosure provide an ultrasound imaging system that improves the speed and efficiency of acquiring accurate ultrasound data that reflects the true anatomy of the optic nerve sheath, that reduces the sensitivity of alignment of the ultrasound probe with respect to the optic nerve sheath, that reduces the need for a skilled operator of the ultrasound probe, that improves the accuracy of optic nerve sheath measurement, and that permits optic nerves sheath measurement in a variety of time-limited and resource-limited settings.

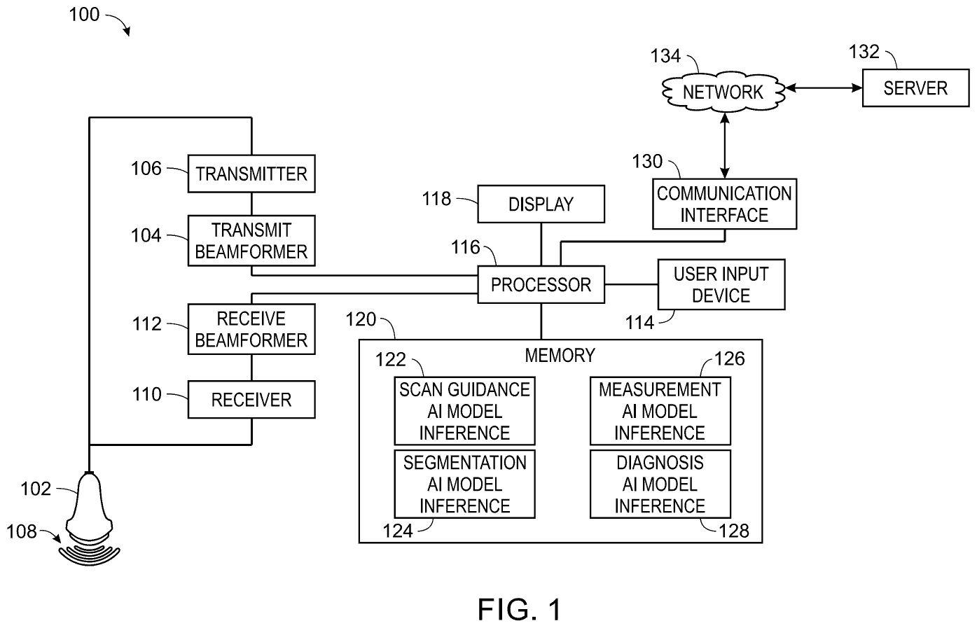

FIG. 1 is a diagram of an ultrasound imaging system 100 according to an embodiment. As shown in FIG. 1, the ultrasound imaging system 100 may include an ultrasound probe 102, a transmit beamformer 104, a transmitter 106, elements 108, a receiver 110, a receive beamformer 112, a user input device 114, a processor 116, a display 118, a memory 120, a scan guidance AI model inference 122, a segmentation AI model inference 124, a measurement AI model inference 126, a diagnosis AI model inference 128, a communication interface 130, a server 132, and a network 134. The foregoing devices may be connected via wired or wireless connections.

The ultrasound probe 102 may be configured to acquire 3D ultrasound data. For example, the ultrasound probe 102 may be a linear probe, a phase array probe, a curved linear probe coupled with a position tracking system, a mechanically steered linear array transducer, a phased array transducer, a curved linear array transducer, an electronically steered 2D transducer array, an electronic 3D (e3D) probe, an electronic 4d (e4D) probe, a low profile wearable patch version of any of the foregoing probes, or the like. According to an embodiment, the ultrasound probe 102 may be configured to emit ultrasound signals suitable for ophthalmic imaging. The ultrasound probe 102 may be configured to generate ultrasound signals, emit the ultrasound signals towards a target location of a subject, receive echo ultrasound signals that are back-scattered from the target location of the subject, generate 3D ultrasound data based on the echo ultrasound signals, and output the 3D ultrasound data. The target location may be an optic nerve sheath, an optic nerve, a retina, or the like. The subject may be a person, an animal, a phantom, or the like.

The transmit beamformer 104 may be configured to apply delay times to electrical signals provided to the elements 108 to focus corresponding ultrasound signals at the target location. The transmitter 106 may be configured to transmit electrical signals to the elements 108 to drive the elements 108 to emit ultrasound signals towards the target location. The elements 108 may be configured to receive the electrical signals from the transmitter 106, convert the electrical signals into ultrasound signals, and emit the ultrasound signals towards the target location. The elements 108 may be configured to receive echo ultrasound signals that are back-scattered by the target location, convert the echo ultrasound signals into electrical signals, and provide the electrical signals to the receiver 110. The receiver 110 may be configured to receive electrical signals from the elements 108, and provide the electrical signals to the receive beamformer 112. The receive beamformer 112 may apply delay times to the electrical signals received from the elements 108.

The user input device 114 may be configured to receive a user input, and provide the user input to the processor 116. For example, the user input device 114 may be a touch screen display, a keyboard, a keypad, a mouse, a button, a switch, a microphone, or the like. Additionally, or alternatively, the user input device 114 may be configured to sense information. For example, the user input device 114 may sense information from an electro-magnetic positioning system, an inertial measurement system, an accelerometer, a gyroscope, an actuator, or the like.

The processor 116 may be configured to perform the operations as described herein. For example, the processor 116 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or the like. The processor 116 may be implemented in hardware, firmware, or a combination of hardware and software. The processor 116 may include one or more processors 116 configured to perform the operations described herein. For example, a single processor 116 may be configured to perform all of the operations described herein. Alternatively, multiple processors 116, collectively, may be configured to perform all of the operations described herein, and each of the multiple processors 116 may be configured to perform a subset of the operations descried herein. For example, a first processor 116 may perform a first subset of the operations described herein, a second processor 116 may be configured to perform a second subset of the operations described herein, etc.

The processor 116 may be configured to control the ultrasound probe 102 to acquire 3D ultrasound data. The processor 116 may be configured to control which of the elements 108 are active, and control the shape of a beam emitted from the ultrasound probe 102. The processor 116 may generate ultrasound images for display. For example, the processor 116 may generate B-mode images, color Doppler images, M-mode images, color M-mode images, or the like.

The display 118 may be configured to display information. For example, the display 118 may be a monitor, a light-emitting diode (LED) display, a cathode ray tube, a projector display, a touchscreen, tablet computer, mobile phone, or the like. The display 118 may display ultrasound images based on the 3D ultrasound data in real-time. For example, the display 118 may display the ultrasound images within one second, two seconds, five seconds, etc., of the 3D ultrasound data being acquired by the ultrasound probe 102.

The memory 120 may be configured to store information and/or instructions for use by the processor 116. The memory 120 may be a non-transitory computer-readable medium. For example, the memory 120 may be a random access memory (RAM), a read only memory (ROM), a flash memory, a magnetic memory, an optical memory, or the like. The memory 120 may be configured to store instructions that, when executed by the processor 116, cause the processor 116 to perform the operations described herein.

The memory 120 may store one or more AI model inferences. For example, the memory 120 may store the scan guidance AI model inference 122 that may be configured to determine a scan quality metric, the segmentation AI model 124 that may be configured to three-dimensionally segment an optic nerve sheath based on 3D ultrasound data, the measurement AI model inference 126 that may be configured to determine a measurement of the optic nerve sheath, and/or the diagnosis AI model inference 128 that may be configured to determine a diagnosis of a subject based on a measurement of the optic nerve sheath of the subject.

The communication interface 130 may be configured to enable the processor 116 to communicate with other devices, such as via a wired connection, a wireless connection, or a combination of wired and wireless connections. For example, the communication interface 130 may include an Ethernet interface, an optical interface, a coaxial interface, an infrared interface, a radio frequency (RF) interface, a universal serial bus (USB) interface, a wireless fidelity (Wi-Fi) interface, a cellular network interface, or the like.

The server 132 may be configured to provide information to the processor 116 and/or the memory 120 via the communication interface 130. For example, the server 132 may be a server, a cloud server, a virtual machine, or the like.

The network 134 may be a local area network (LAN), a wide area network (WAN), a metropolitan area network (MAN), a cellular network, a private network, an ad hoc network, an intranet, the Internet, a fiber optic-based network, or the like, and/or a combination of these or other types of networks.

The number and arrangement of the devices of the ultrasound imaging system 100 shown in FIG. 1 are provided as an example. In practice, the ultrasound imaging system 100 may include additional devices, fewer devices, different devices, or differently arranged devices than those shown in FIG. 1. Additionally, or alternatively, a set of devices (e.g., one or more devices) of the ultrasound imaging system 100 may perform one or more functions described as being performed by another set of devices of the ultrasound imaging system 100.

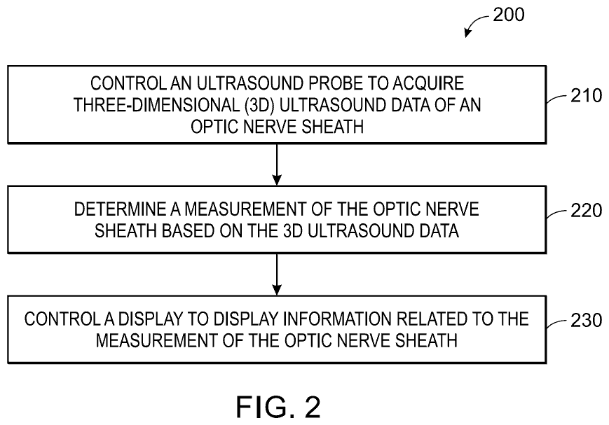

FIG. 2 is a flowchart of an example process 200 for determining an optic nerve sheath measurement according to an embodiment. The ultrasound imaging system 100 may be configured to perform the operations of the process 200.

As shown in FIG. 2, the process 200 may include controlling an ultrasound probe to acquire three-dimensional (3D) ultrasound data of an optic nerve sheath (operation 210). The 3D ultrasound data may correspond to the ocular anatomy of the subject. That is, the 3D ultrasound data may include the optic nerve, the optic nerve sheath, the lens, the iris, the cornea, the retina, the macula, the optic disc, the vitreous body, the choroid, etc. The processor 116 may generate ultrasound images based on the 3D ultrasound data. The ultrasound images may be 3D images, 2D images, single plane images, bi-plane images, three-plane images, multi-plane images, or the like. The ultrasound images may correspond to various anatomical planes (e.g., sagittal, coronal, and transverse) of the ocular anatomy. Further, the ultrasound images may correspond to a long-axis plane of the ocular anatomy or a short-axis plane of the ocular anatomy. The long-axis plane of the ocular anatomy may be a plane in which the optic nerve sheath appears substantially tubular. The short-axis plane of the ocular anatomy may be a plane in which the optic nerve sheath appears substantially circular or substantially elliptical.

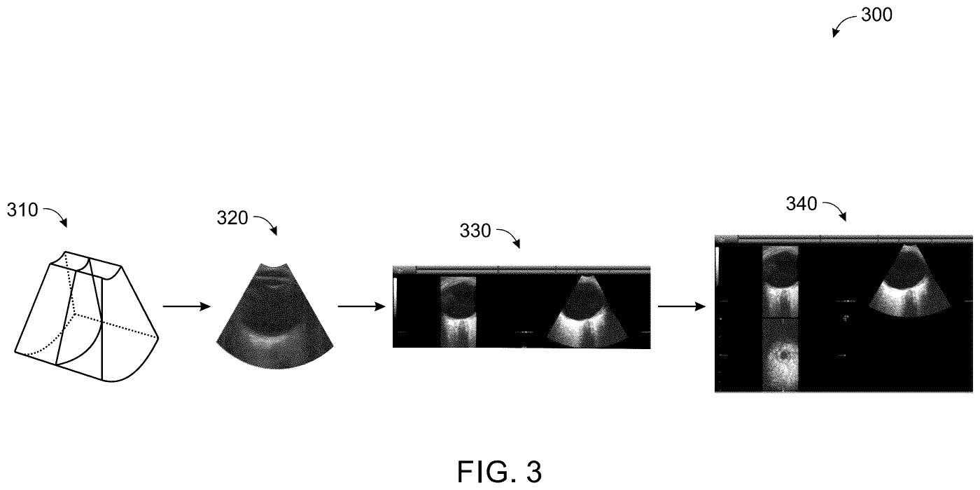

According to an embodiment, and to assist the operator of the ultrasound probe 102 to acquire the 3D ultrasound data, the processor 116 may control the display 118 to display preview ultrasound images in real-time. FIG. 3 is a diagram 300 of displaying preview ultrasound images in real-time according to an embodiment. As shown in FIG. 3, the acquired 3D ultrasound data may be represented as a volume 310. The processor 116 may generate various preview ultrasound images corresponding to various planes of the ocular anatomy based on the 3D ultrasound data. For example, as shown, the preview ultrasound image 320 may correspond to the transverse plane and a long-axis plane of the ocular anatomy. The processor 116 may control the display 118 to display bi-plane preview ultrasound images 330 and preview ultrasound images 340 corresponding to various anatomical planes of the ocular anatomy, and the long-axis plane and short-axis plane of the ocular anatomy in real-time. For example, the processor 116 may control the display 118 to display preview ultrasound images such as single plane images, bi-plane images, three-plane images, multi-plane images, curved plane images, 3D renderings or the like, in real-time.

According to another embodiment, and to also assist the operator of the ultrasound probe 102 to acquire the 3D ultrasound data, the processor 116 may generate scan guidance information, and control the display 118 to display the scan guidance information to assist or guide the operator in positioning the ultrasound probe 102 to acquire the 3D ultrasound data for determining the measurement of the optic nerve sheath.

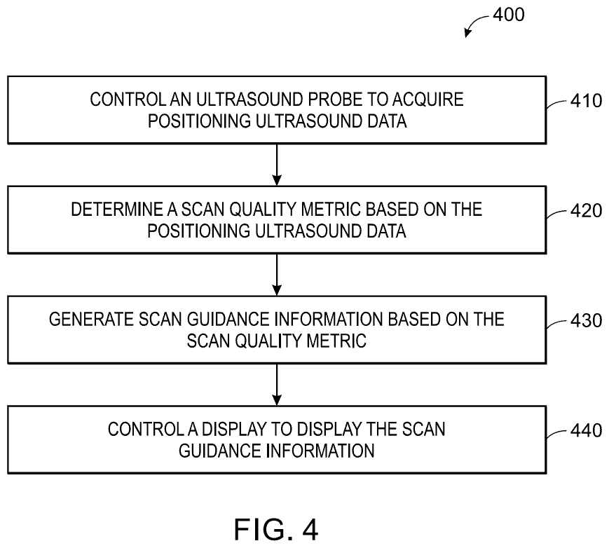

FIG. 4 is a flowchart of an example process 400 for generating scan guidance information according to an embodiment. The ultrasound imaging system 100 may be configured to perform the operations of the process 400.

As shown in FIG. 4, the process 400 may include controlling an ultrasound probe to acquire positioning ultrasound data (operation 410). The positioning ultrasound data may be ultrasound data that is used by the processor 116 to generate scan guidance information. According to an embodiment, the processor 116 may control the display 118 to display ultrasound images corresponding to the positioning ultrasound data in real-time. According to another embodiment, the processor 116 may refrain from controlling the display 118 to display ultrasound images corresponding to the positioning ultrasound data. For instance, the processor 116 may utilize the positioning ultrasound data to generate scan guidance information and the processor 116 may control the display 118 to scan guidance information, and might not control the display 118 to display ultrasound images corresponding to the positioning ultrasound data.

As further shown in FIG. 4, the process 400 may include determining a scan quality metric based on the positioning ultrasound data (operation 420). The scan quality metric may indicate a quality of 3D ultrasound data for determining an optic nerve sheath measurement. For instance, 3D ultrasound data having a low scan quality metric may result in an inaccurate, or less accurate, optic nerve sheath measurement, whereas 3D ultrasound data having a high scan quality metric may result in an accurate, or more accurate, optic nerve sheath measurement.

According to an embodiment, the processor 116 may determine the scan quality metric based on one or more image quality properties of the positioning ultrasound data. The image quality properties may be a spatial resolution, a contrast resolution, probe motion parameter, a noise parameter, an artifact parameter, or the like. According to another embodiment, the processor 116 may determine the scan quality metric based on a degree of alignment between an anatomical structure of the ocular anatomy of the subject and an alignment reference. The anatomical structure may be a centerline of the optic nerve, an optic nerve, an optic nerve sheath, a landmark of the retina, or the like. The alignment reference may be a focal point of an ultrasound beam, a centerline of the acquisition volume for the 3D ultrasound data, or the like. According to another embodiment, the processor 116 may determine the scan quality metric based on the scan guidance AI model inference 122. For example, the processor 116 may input the positioning ultrasound data into the scan guidance AI model inference 122, and determine the scan quality metric based on an output of the scan guidance AI model inference 122.

As further shown in FIG. 4, the process 400 may include generating scan guidance information based on the scan quality metric (operation 430), and controlling the display to display the scan guidance information (operation 440). The scan guidance information may be information that assists the operator to position the ultrasound probe 102 with respect to the subject. For example, the scan guidance information may indicate whether a position of the ultrasound probe 102 is suitable, or optimal, for acquiring 3D ultrasound data for determining a measurement of an optic nerve sheath. An operator of the ultrasound probe 102 can utilize the scan guidance information to position the ultrasound probe 102 with respect to the subject. For instance, if the scan guidance information indicates that an improved, or optimal, position of the ultrasound probe 102 can be achieved by moving the ultrasound probe 102 from a current position, then the operator may re-position the ultrasound probe 102 from the current position towards the improved, or optimal, position.

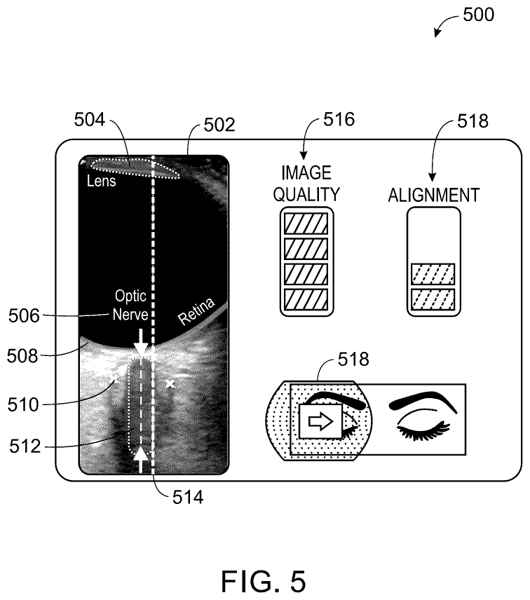

According to an embodiment, the scan guidance information may include an ultrasound image. FIG. 5 is a diagram of a display 500 of scan guidance information according to an embodiment. For example, as shown in FIG. 5, the scan guidance information may include a diagram 518 indicating where to position the ultrasound probe 102 on the subject for the current exam. For example, as shown in FIG. 5, the scan guidance information may include an ultrasound image 502. Additionally, the scan guidance information may include an identifier of an anatomical structure. For example, as shown in FIG. 5, the scan guidance information may include an identifier 504 of a lens, and an identifier 506 of an optic nerve, and an identifier 508 of a retina. Additionally, or alternatively, the scan guidance information may include an indicator of a measurement position at which the processor 116 is configured to determine a measurement of the optic nerve sheath using 3D ultrasound data. For example, as shown in FIG. 5, the scan guidance information may include an indicator 510 of a measurement position at which the processor 116 is configured to determine a measurement of the optic nerve sheath using 3D ultrasound data. Additionally, or alternatively, the scan guidance information may include an indicator of an anatomical structure. For example, as shown in FIG. 5, the scan guidance information may include an indicator 512 of a centerline of an optic nerve. Additionally, or alternatively, the scan guidance information may include an indicator of an alignment reference. For example, as shown in FIG. 5, the scan guidance information may include an indicator 514 of a centerline of the acquisition volume for the 3D ultrasound data 502.

According to an embodiment, the scan guidance information may be a preview ultrasound image. For example, the processor 116 may generate preview ultrasound images based on the positioning ultrasound data, and control the display 118 to display the preview ultrasound image in real-time. The preview ultrasound images may be single plane images, bi-plane images, three-plane images, multi-plane images, curved plane images, 3D renderings, or the like.

According to an embodiment, the scan guidance information may be an indicator of an image quality. For example, the indicator may be a discrete value of an image quality (e.g., 90%, 50%, 7, 10, etc.), a representation of an image quality (e.g., a variable number of bars, a variable brightness, a variable status, or the like), or the like. As an example, and as shown in FIG. 5, the scan guidance information may include an indicator 516 of image quality that is a representation in the form of bars.

According to an embodiment, the scan guidance information may be an indicator of a level of alignment between an anatomical structure and an alignment reference. For example, the indicator may be a discrete value of a level of alignment (e.g., 90%, 50%, 7, 10, etc.), a representation of a level of alignment (e.g., a variable number of bars, a variable brightness, a variable status, or the like), or the like. As an example, and as shown in FIG. 5, the scan guidance information may include an indicator 518 of a level of alignment that is a representation in the form of bars.

According to an embodiment, the scan guidance information may be an indicator of a direction in which to move the ultrasound probe 102 to acquire improved, or optimal, 3D ultrasound data for determining a measurement of the optic nerve sheath. For example, the indicator may indicate whether to move the ultrasound probe 102 vertically, move the ultrasound probe 102 horizontally, tilt the ultrasound probe 102, rotate the ultrasound probe 102, or the like. As an example, and as shown in FIG. 5, the scan guidance information may include an indicator 518 that indicates that the ultrasound probe 102 should be moved horizontally towards a center of the subject in order to achieve a position that provides for the acquisition of improved, or optimal, 3D ultrasound data for determining a measurement of the optic nerve sheath.

The processor 116 may control the display 118 to display the scan guidance information, generate updated scan guidance information based on updated positioning ultrasound data as the operator moves the ultrasound probe 102 based on the scan guidance information to acquire the updated positioning ultrasound data, and control the display 118 to display the updated scan guidance information. In this way, an operator of the ultrasound probe 102 may ascertain whether a position of the ultrasound probe 102 is suitable for acquiring 3D ultrasound data for determining a measurement of the optic nerve sheath, and may move the ultrasound probe 102 to an improved, or optimal, position based on the scan guidance information indicating that the improved, or optimal, position exists. Although FIG. 4 shows example operations of the process 400, in some embodiments, the process 400 may include additional operations, fewer operations, different operations, or differently arranged operations than those depicted in FIG. 4. Additionally, or alternatively, two or more of the operations of the process 400 may be performed in parallel.

Referring to FIG. 2, and according to an embodiment, the processor 116 may control the ultrasound probe 102 to acquire the 3D ultrasound data for determining a measurement of the optic nerve sheath based on a user input. For example, an operator of the ultrasound probe 102 may interact with the user input device 114 to provide a user input to the processor 116, and the processor 116 may control the ultrasound probe 102 to acquire the 3D ultrasound data based on the user input.

According to another embodiment, the processor 116 may control the ultrasound probe 102 to automatically acquire the 3D ultrasound data for determining a measurement of the optic nerve sheath. For instance, the processor 116 may control the ultrasound probe 102 to automatically acquire the 3D ultrasound data without having received a user input to acquire the 3D ultrasound data.

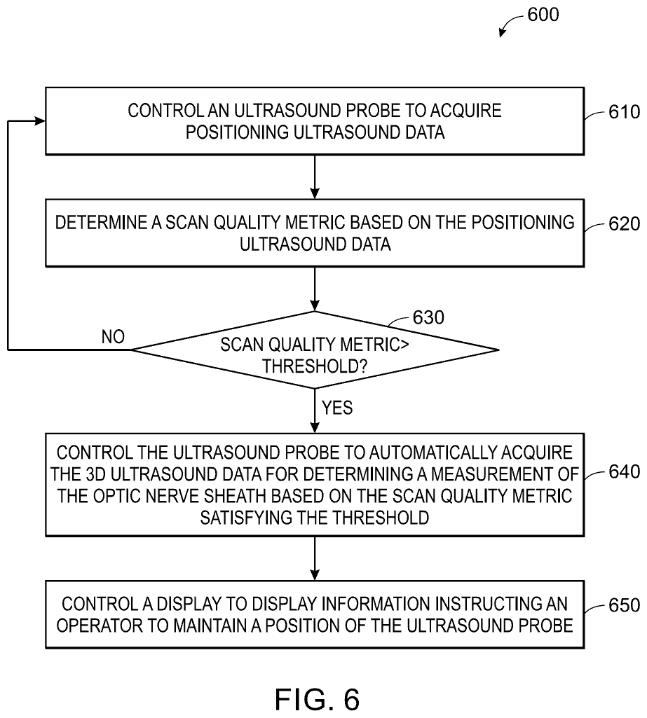

FIG. 6 is a flowchart of an example process 600 for controlling an ultrasound probe to automatically acquire 3D ultrasound data based on a scan quality metric according to an embodiment. The ultrasound imaging system 100 may be configured to perform the operations of the process 600.

As shown in FIG. 6, the process 600 may include controlling an ultrasound probe to acquire positioning ultrasound data (operation 610), and determining a scan quality metric based on the positioning ultrasound data (operation 620). For example, the processor 116 may control the ultrasound probe 102 to acquire positioning ultrasound data in a similar manner as described above in connection with operation 410 of FIG. 4, and may determine a scan quality metric based on the positioning ultrasound data in a similar manner as described above in connection with operation 420 of FIG. 4.

As further shown in FIG. 6, the process 600 may include determining whether the scan quality metric satisfies a threshold (operation 630). The threshold may indicate a scan quality metric that results in 3D ultrasound data that is suitable for providing an accurate, or optimal, measurement of the optic nerve sheath. The processor 116 may compare the scan quality metric to the threshold, and determine whether the scan quality metric satisfies the threshold based on the scan quality metric being greater than or equal to the threshold.

As further shown in FIG. 6, if the scan quality metric does not satisfy the threshold (operation 630—NO), then the process 600 may return to operation 610. However, as further shown in FIG. 6, if scan quality metric does satisfy the threshold (operation 630—YES), then the process 600 may include controlling the ultrasound probe to automatically acquire the 3D ultrasound data (operation 640). For example the processor 116 may control the ultrasound probe 102 to automatically acquire the 3D ultrasound data for determining a measurement of the optic nerve sheath based on the scan quality metric satisfying the threshold.

As further shown in FIG. 6, the process 600 may include controlling a display to display information instructing an operator to maintain a position of the ultrasound probe (operation 650). For example, the processor 116 may control the display 118 to display information that instructs the operator to maintain a position of the ultrasound probe 102 while the ultrasound probe 102 acquires the 3D ultrasound data. As an example, the information may instruct the operator to keep the ultrasound probe 102 stationary.

Although FIG. 6 shows example operations of the process 600, in some embodiments, the process 600 may include additional operations, fewer operations, different operations, or differently arranged operations than those depicted in FIG. 6. Additionally, or alternatively, two or more of the operations of the process 600 may be performed in parallel.

Referring to FIG. 2, the ultrasound probe 102 may acquire the 3D ultrasound data. For example, the operator of ultrasound probe 102 may position the ultrasound probe 102 with respect to the subject, and the processor 116 may control the ultrasound probe 102 to acquire the 3D ultrasound data based on a user input or automatically.

As further shown in FIG. 2, the process 200 may include determining a measurement of the optic nerve sheath based on the 3D ultrasound data (operation 220). The measurement may be a diameter of the optic nerve sheath, a radius of the optic nerve sheath, a cross-sectional area of the optic nerve sheath, a volume of a longitudinal segment of the optic nerve sheath, or the like. The measurement of the optic nerve sheath may correspond to the exterior boundary of the optic nerve sheath. Alternatively, the measurement of the optic nerve sheath may correspond to the interior boundary. Alternatively, the measurement may correspond to a center of the optic nerve sheath.

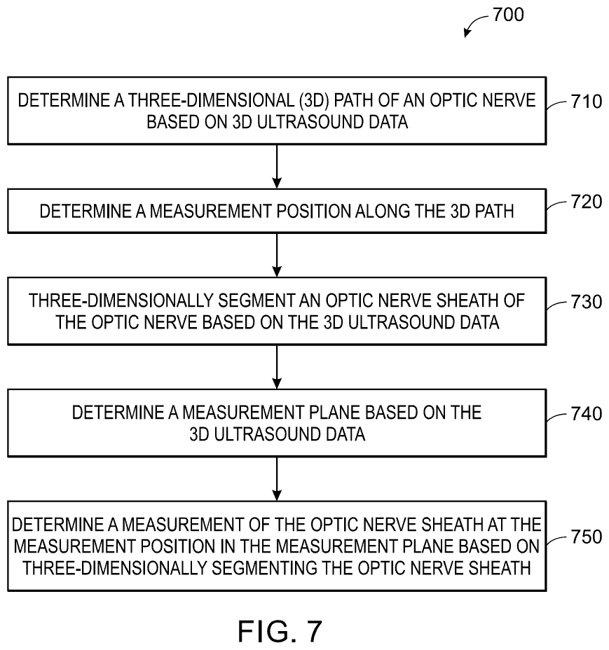

FIG. 7 is a flowchart of an example process 700 for determining a measurement of the optic nerve sheath based on 3D ultrasound data according to an embodiment. The ultrasound imaging system 100 may be configured to perform the operations of the process 700.

As shown in FIG. 7, the process 700 may include determining a 3D path of an optic nerve based on 3D ultrasound data (operation 710). The 3D path of the optic nerve may identify the position and curvature of the optic nerve. The processor 116 may determine the 3D path of the optic nerve using an image analysis technique in association with the 3D ultrasound data.

According to an embodiment, the processor 116 may determine a centerline of the optic nerve based on the 3D path of the optic nerve. The processor 116 may determine the centerline using an image analysis technique in association with the 3D ultrasound data. According to an embodiment, the processor 116 may control the ultrasound probe 102 to acquire 3D B-mode ultrasound data, and determine the centerline of the optic nerve based on the 3D B-mode ultrasound data. According to an embodiment, the processor 116 may control the ultrasound probe 102 to acquire 3D color flow Doppler ultrasound data, and determine the centerline of the optic nerve based on the 3D color flow Doppler ultrasound data. The technique to determine the 3D path of the optic nerve may include region growing segmentation, eigen analysis to identify tubular structures, or the like.

As further shown in FIG. 7, the process 700 may include determining a measurement position along the 3D path (operation 720). The measurement position may be a position along the 3D path of the optic nerve at which the processor 116 is configured to determine a measurement of the optic nerve sheath. The measurement position may be a position that is located at a predetermined distance from an anatomical structure. As examples, the measurement position may be 3 millimeters (mm) from a retina, 4 mm from the retina, 5 mm from the retina, etc. The measurement position may be predetermined based on clinical guidelines, may be input via an operator, may be arbitrarily selected, or the like. The processor 116 may determine the measurement position based on determining a position of the anatomical structure, and based on determining a position that is located at the predetermined distance from the anatomical structure along the 3D path. For instance, the processor 116 may determine the measurement position as the position that is located at the predetermined distance from the anatomical structure along the 3D path.

As further shown in FIG. 7, the process 700 may include three-dimensionally segmenting an optic nerve sheath of the optic nerve based on the 3D ultrasound data (operation 730). For example, the processor 116 may obtain a three-dimensionally segmented optic nerve sheath based on three-dimensionally segmenting the optic nerve sheath. The processor 116 may three-dimensionally segment a portion of the optic nerve sheath. For example, the processor 116 may three-dimensionally segment an exterior boundary of the optic nerve sheath. Additionally, or alternatively, the processor 116 may three-dimensionally segment an interior boundary of the optic nerve sheath. Additionally, or alternatively, the processor 116 may segment any other portion of the optic nerve sheath.

According to an embodiment, the processor 116 may three-dimensionally segment the optic nerve sheath using a segmentation technique in association with the 3D ultrasound data. The segmentation technique may include edge detection, region-based segmentation, clustering-based segmentation, surface models that deforms to edges, neural network-based segmentation, or the like. According to an embodiment, the processor 116 may generate plurality of ultrasound images based on the 3D ultrasound data, and three-dimensionally segment the optic nerve sheath using the ultrasound images.

According to another embodiment, the processor 116 may three-dimensionally segment the optic nerve sheath using the segmentation AI model inference 124. For example, the processor 116 may input the 3D ultrasound data into the segmentation AI model inference 124, and obtain the three-dimensionally segmented optic nerve sheath based on an output of the segmentation AI model inference 124. In this case, the segmentation AI model inference 124 may be configured to three-dimensionally segment the optic nerve sheath directly from the 3D ultrasound data.

According to another embodiment, the processor 116 may three-dimensionally segment the optic nerve sheath using a structural model. The structural model may be a circular cylinder, a curved cylinder having a smoothly varying radius, an elliptical cylinder, or the like. The processor 116 may use an image analysis technique and the structural model to three-dimensionally segment the optic nerve sheath. By using the structural model, the processor 116 may regularize, or adjust, the optic nerve sheath in the ultrasound image. For example, the processor 116 may generate an ultrasound image in which the optic nerve sheath appears substantially straight despite the physical optic nerve sheath being curved. The processor 116 may align the various planes with respect to the center of the optic nerve. That is, the intersection of the planes may be the center of the optic nerve. As another example, the processor 116 may control the display 118 to display a preview ultrasound image that in follows a 3D path of an optic nerve. In this case, a single plane image might not show the entirety of the optic nerve because of the curvature of the optic nerve. As such, by controlling the display 118 to display the ultrasound preview image corresponding to the curved plane, the processor 116 may facilitate the acquisition of more accurate ultrasound images for determining the measurement of the optic nerve sheath.

According to another embodiment, the processor 116 may segment the optic nerve sheath using a 2D ultrasound image. For example, the processor 116 may generate a 2D ultrasound image based on the 3D ultrasound data, and segment the optic nerve sheath using the 2D ultrasound image.

As further shown in FIG. 7, the process 700 may include determining a measurement plane based on the 3D ultrasound data (operation 740). The measurement plane may be a plane of the 3D ultrasound data in which the processor 116 is configured to determine a measurement of the optic nerve sheath. For example, the measurement plane may be an anatomical plane of the ocular anatomy, and may be a long-axis plane of the ocular anatomy or a short-axis plane of the ocular anatomy.

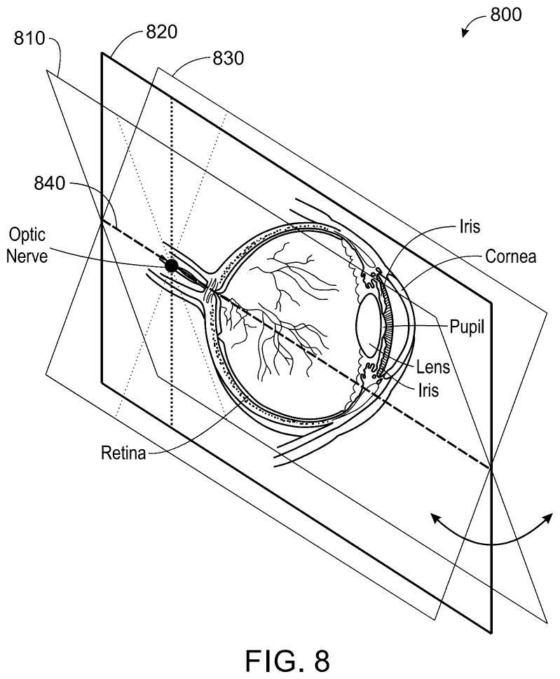

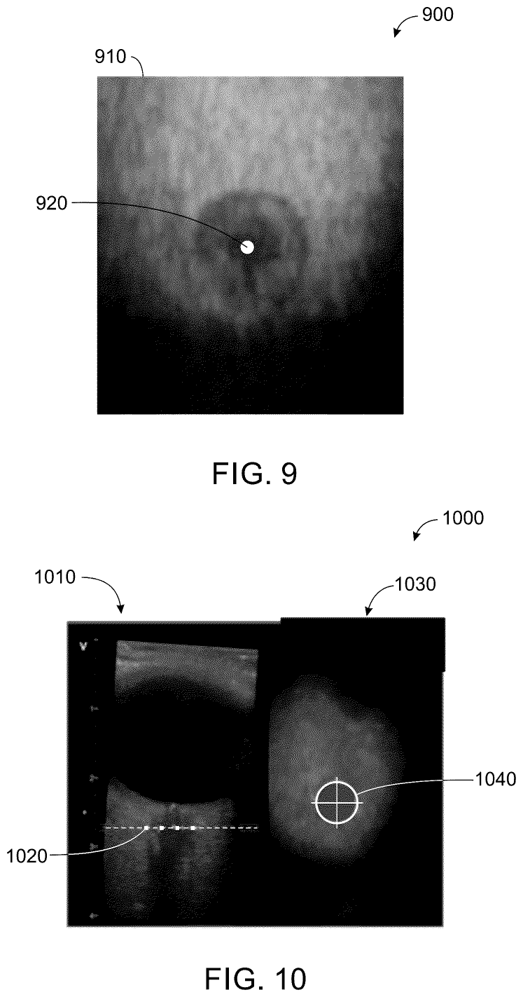

The processor 116 may determine a measurement plane based on the 3D path of the optic nerve. For example, the processor 116 may determine a measurement plane based on a centerline of the optic nerve, a curvature of the optic nerve, or the like. According to an embodiment, the processor 116 may determine a long-axis measurement plane that is planar with the centerline of the optic nerve. FIG. 8 is a diagram 800 of long-axis measurement planes according to an embodiment. As shown in FIG. 8, the processor 116 may determine a first measurement plane 810, a second measurement plane 820, and a third measurement plane 830 that are each planar with a centerline 840 of the optic nerve. In this way, the processor 116 may determine a more accurate measurement of the optic nerve sheath because the measurement plane is provided at a centerline of the optic nerve. According to another embodiment, the processor 116 may determine a short-axis measurement plane that is orthogonal to a centerline of the optic nerve. Further, the processor 116 may determine the short-axis measurement plane that is located at the measurement position. That is, the measurement plane may be located at a predetermined distance from an anatomical structure. FIG. 9 is a diagram 900 of a short-axis measurement plane according to an embodiment. As shown in FIG. 9, the processor 116 may determine a measurement plane 910 that is orthogonal to a centerline 920 of the optic nerve. In this way, the processor 116 may determine a more accurate measurement of the optic nerve sheath because the measurement plane is provided orthogonal to the optic nerve sheath.

As further shown in FIG. 7, the process 700 may include determining a measurement of the optic nerve sheath at the measurement position in the measurement plane based on three-dimensionally segmenting the optic nerve sheath (operation 750). For example, the processor 116 may determine a measurement of the optic nerve sheath at the measurement position in the measurement plane based on the three-dimensionally segmented optic nerve sheath.

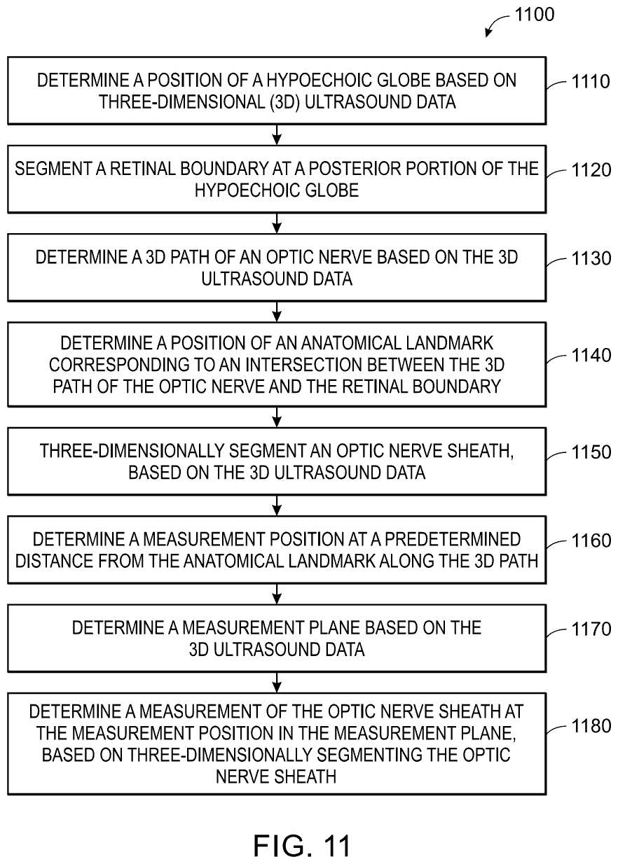

According to an embodiment, the processor 116 may determine a measurement of the optic nerve sheath in a long-axis plane. For example, the processor 116 may determine a measurement of the optic nerve sheath orthogonally to the centerline of the optic nerve at a measurement position in a long-axis plane. The processor 116 may determine a distance between two portions of the optic nerve sheath, and determine a diameter, a radius, an area, a volume, etc., of the optic nerve sheath based on the distance. FIG. 10 is a diagram 1000 of measurements of the optic nerve sheath according to an embodiment. As shown in FIG. 10, the processor 116 may determine a diameter of the optic nerve sheath in a long-axis plane 1010 by measuring a distance 1020 between the exterior boundaries of the optic nerve sheath.

According to another embodiment, the processor 116 may determine a measurement of the optic nerve sheath in a short-axis plane. For example, the processor 116 may determine a measurement of the optic nerve sheath orthogonally to the centerline of the optic nerve in a short-axis plane. In this case, the short-axis plane may correspond to the measurement position. The processor 116 may determine a distance between two portions of the optic nerve sheath, and determine a diameter, a radius, an area, a volume, etc., of the optic nerve sheath based on the distance. Additionally, or alternatively, the processor 116 may use a structural model (e.g., a circle, an ellipse, or the like) to determine the measurement of the optic nerve sheath. For example, the processor 116 may fit the structural model to correspond to the optic nerve sheath, and determine the measurement of the optic nerve sheath based on a measurement of the structural model. As shown in FIG. 10, the processor 116 may determine an area of the optic nerve sheath in a short-axis plane 1030 by fitting a geometrical model 1040 to the optic nerve sheath. Although FIG. 7 shows example operations of the process 700, in some embodiments, the process 700 may include additional operations, fewer operations, different operations, or differently arranged operations than those depicted in FIG. 7. Additionally, or alternatively, two or more of the operations of the process 700 may be performed in parallel.

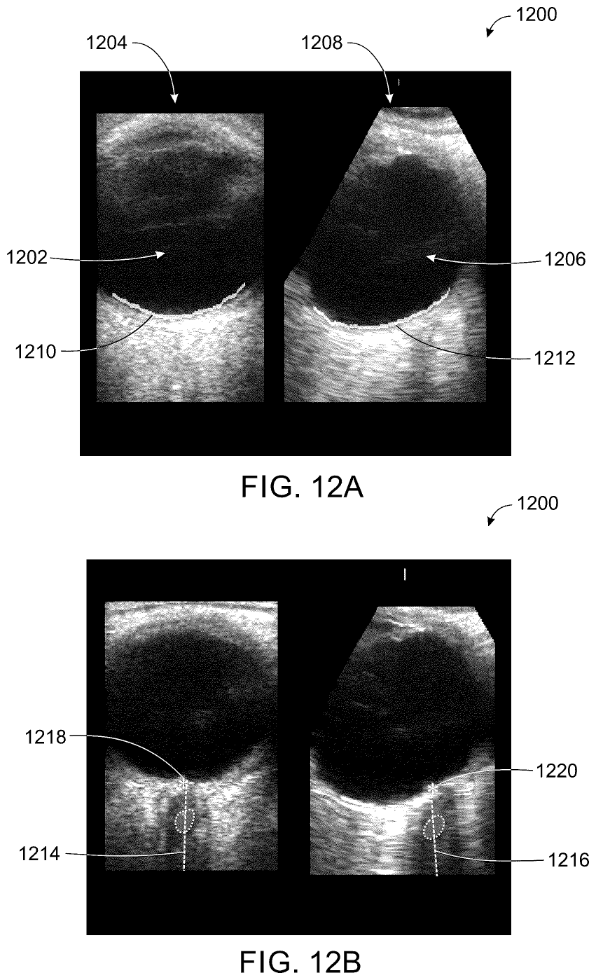

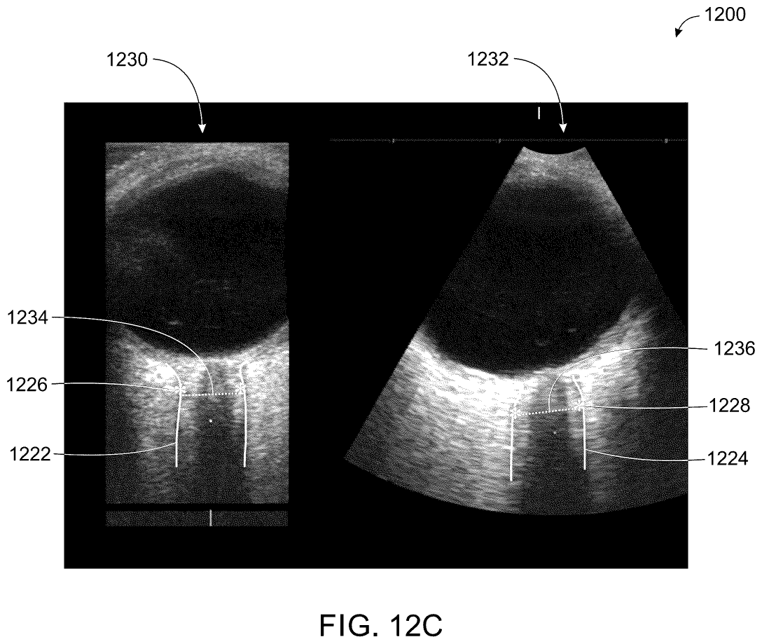

FIG. 11 is a flowchart of an example process 1100 for determining a measurement of the optic nerve sheath based on 3D ultrasound data according to an embodiment. The ultrasound imaging system 100 may be configured to perform the operations of the process 1100. FIGS. 12A-12C are diagrams 1100 of ultrasound images for determining a measurement of the optic nerve sheath based on 3D ultrasound data according to an embodiment.

As shown in FIG. 11, the process 1100 may include determining a position of a hypoechoic globe based on 3D ultrasound data (operation 1110). For example, as shown in FIG. 12A, the processor 116 may determine a position of a hypoechoic globe 1202 in a transverse plane 1204 of the ultrasound data, and determine a position of a hypoechoic globe 1206 in a sagittal plane 1208 of the 3D ultrasound data. The hypoechoic globe 1202 may correspond to a vitreous body of the ocular anatomy.

As further shown in FIG. 11, the process 1100 may include segmenting a retinal boundary at a posterior portion of the hypoechoic globe (operation 1120). For example, as shown in FIG. 12A, the processor 116 may segment a retinal boundary 1210 at a posterior portion of the hypoechoic globe 1202, and may segment a retinal boundary 1212 at a posterior portion of the hypoechoic globe 1206.

As further shown in FIG. 11, the process 1100 may include determining a 3D path of an optic nerve based on the 3D ultrasound data (operation 1130). For example, as shown in FIG. 12B, the processor 116 may determine a 3D path 1214 of an optic nerve based on the ultrasound data, and may determine a 3D path 1216 of the optic nerve based on the ultrasound data.

As further shown in FIG. 11, the process 1100 may include determining a position of an anatomical landmark corresponding to an intersection between the 3D path of the optic nerve and the retinal boundary (operation 1140). For example, as shown in FIG. 12B, the processor 116 may determine a position of an anatomical landmark 1218 corresponding to an intersection between the 3D path 1214 of the optic nerve and the retinal boundary 1210, and may determine a position of an anatomical landmark 1220 corresponding to an intersection between the 3D path 1216 of the optic nerve and the retinal boundary 1212.

As further shown in FIG. 11, the process 1100 may include three-dimensionally segmenting the optic nerve sheath based on the 3D ultrasound data (operation 1150). For example, as shown in FIG. 12C, the processor 116 may three-dimensionally segment the optic nerve sheath based on the 3D ultrasound data to obtain a three-dimensionally segmented optic nerve sheath 1222 and a three-dimensionally segmented optic nerve sheath 1224.

As further shown in FIG. 11, the process 1100 may include determining a measurement position at a predetermined distance from the anatomical landmark along the 3D path (operation 1160). For example, as shown in FIG. 12C, the processor 116 may determine a measurement position 1226 at a predetermined distance from the anatomical landmark 1218 along the 3D path 1214, and may determine a measurement position 1228 at a predetermined distance from the anatomical landmark 1220 along the 3D path 1216.

As further shown in FIG. 11, the process 1100 may include determining a measurement plane based on the 3D ultrasound data (operation 1170). For example, as shown in FIG. 12C, the processor 116 may determine a measurement plane 1230 corresponding to the transverse plane 1204, and may determine a measurement plane 1232 corresponding to the sagittal plane 1208 based on the ultrasound data.

As further shown in FIG. 11, the process 1100 may include determining a measurement of the optic nerve sheath at the measurement position in the measurement plane based on three-dimensionally segmenting the optic nerve sheath (operation 1180). For example, as shown in FIG. 12C, the processor 116 may determine a measurement 1234 of the optic nerve sheath at the measurement position 1226 in the measurement plane 1230 based on the three-dimensionally segmented optic nerve sheath 1222, and may determine a measurement 1236 of the optic nerve sheath at the measurement position 1228 in the measurement plane 1232 based on the three-dimensionally segmented optic nerve sheath 1224.

Although FIG. 11 shows example operations of the process 1100, in some embodiments, the process 1100 may include additional operations, fewer operations, different operations, or differently arranged operations than those depicted in FIG. 11. Additionally, or alternatively, two or more of the operations of the process 1100 may be performed in parallel.

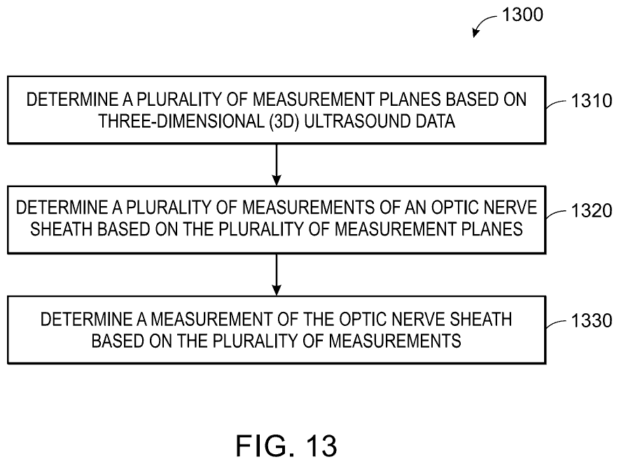

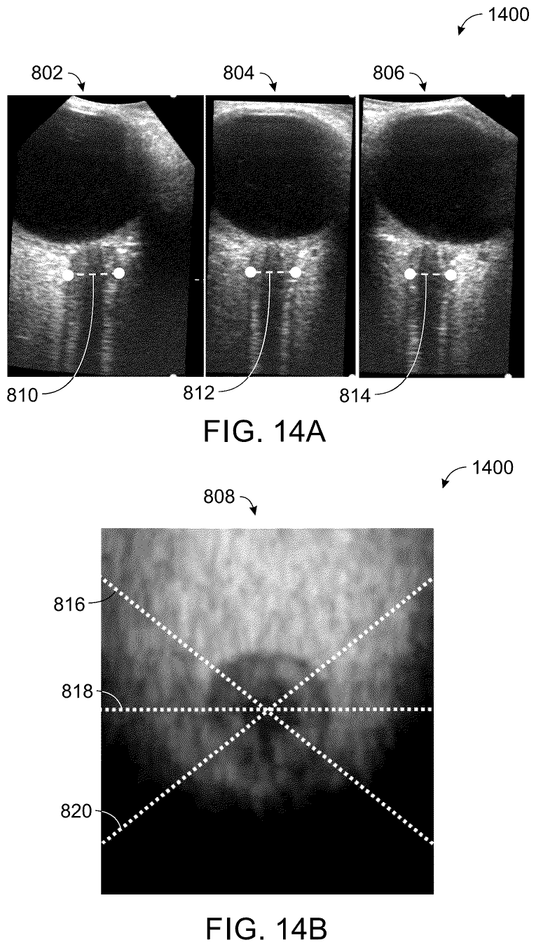

FIG. 13 is a flowchart of an example process 1300 for determining a measurement of the optic nerve sheath based on 3D ultrasound data according to an embodiment. The ultrasound imaging system 100 may be configured to perform the operations of the process 1300. FIGS. 14A and 14B are diagrams 1400 of ultrasound images for determining a measurement of the optic nerve sheath based on 3D ultrasound data according to an embodiment.

As shown in FIG. 13, the process 1300 may include determining a plurality of measurement planes based on 3D ultrasound data (operation 1310). For example, the processor 116 may determine a plurality of measurement planes in a similar manner as described above in connection with operation 740 of FIG. 7. As shown in FIG. 14A, the processor 116 may determine a measurement plane 802, a measurement plane 804, and a measurement plane 806 that each correspond to a long-axis plane of the 3D ultrasound data. As shown in FIG. 14B, the processor 116 may determine a measurement plane 808 that corresponds to a short-axis plane of the 3D ultrasound data.

As further shown in FIG. 13, the process 1300 may include determining a plurality of measurements of an optic nerve sheath based on the plurality of measurement planes (operation 1320). For example, the processor 116 may determine a plurality of measurements of the optic nerve sheath in a similar manner as described above in connection with operation 750 of FIG. 7. As shown in FIG. 14A, the processor 116 may determine a measurement 810 in the measurement plane 802, a measurement 812 in the measurement plane 804, and a measurement 814 in the measurement plane 806. As shown in FIG. 14B, the processor 116 may determine a measurement 816, a measurement 818, and a measurement 820 in the measurement plane 808.

As further shown in FIG. 13, the process 1300 may include determining a measurement of the optic nerve sheath based on the plurality of measurements (operation 1330). The measurement may be an average measurement of the plurality of measurements, the measurement having the greatest value, the measurement having the lowest value, a measurement having an intermediate value, or the like.

According to an embodiment, the processor 116 may apply a respective weight value to each of the plurality of measurements, and determine the measurement of the optic nerve sheath based on applying the respective weight values. The processor 116 may determine a weight value for a measurement based on a scan quality metric of the underlying 3D ultrasound data that was used to determine the measurement. Additionally, or alternatively, the processor 116 may determine a weight value based on a quality of the three-dimensionally segmented optic nerve sheath. Additionally, or alternatively, the processor 116 may determine a weight value based on predetermined weight values to apply to various respective measurement planes.

According to an embodiment, the processor 116 may determine the measurement based on a plurality of measurements corresponding to a plurality of measurement planes of a same axis view of the optic nerve sheath. For example, the processor 116 may determine the measurement based on a plurality of measurements in a plurality of measurement planes corresponding to a long-axis view of the optic nerve sheath. As another example, the processor 116 may determine the measurement based on a plurality of measurements in a plurality of measurement planes corresponding to a short-axis view of the optic nerve sheath. According to another embodiment, the processor 116 may determine the measurement based on a plurality of measurements corresponding to a plurality of measurement planes of different axis views of the optic nerve sheath. For example, the processor 116 may determine the measurement based on a measurement in a measurement plane corresponding to a long-axis view of the optic nerve sheath and a measurement in a measurement plane corresponding to a short-axis view of the optic nerve sheath.

Although FIG. 13 shows example operations of the process 1300, in some embodiments, the process 1300 may include additional operations, fewer operations, different operations, or differently arranged operations than those depicted in FIG. 13. Additionally, or alternatively, two or more of the operations of the process 1300 may be performed in parallel.

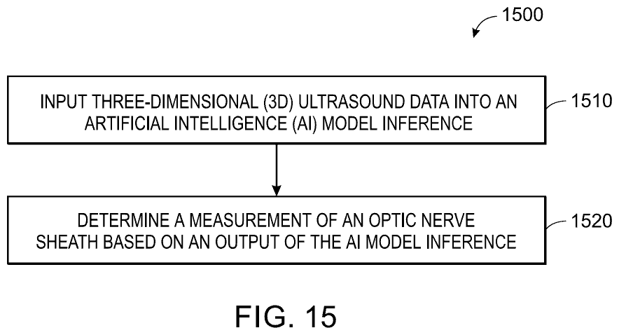

FIG. 15 is a flowchart of an example process 1500 for determining a measurement of the optic nerve sheath based on 3D ultrasound data according to an embodiment. The ultrasound imaging system 100 may be configured to perform the operations of the process 1500.

As shown in FIG. 15, the process 1500 may include inputting 3D ultrasound data into an AI model inference (operation 1510), and determining a measurement of an optic nerve sheath based on an output of the AI model inference (operation 1520). For example, the processor 116 may input 3D ultrasound data into the measurement AI model inference 126, and determine a measurement of the optic nerve sheath based on an output of the measurement AI model inference 126. The measurement AI model inference 126 may be configured to determine a measurement of an optic nerve sheath directly from the 3D ultrasound data. That is, the measurement AI model inference 126 may be configured to determine the measurement of the optic nerve sheath without having performed any preliminary operations, such as segmentation. Although FIG. 15 shows example operations of the process 1500, in some embodiments, the process 1500 may include additional operations, fewer operations, different operations, or differently arranged operations than those depicted in FIG. 15. Additionally, or alternatively, two or more of the operations of the process 1500 may be performed in parallel.

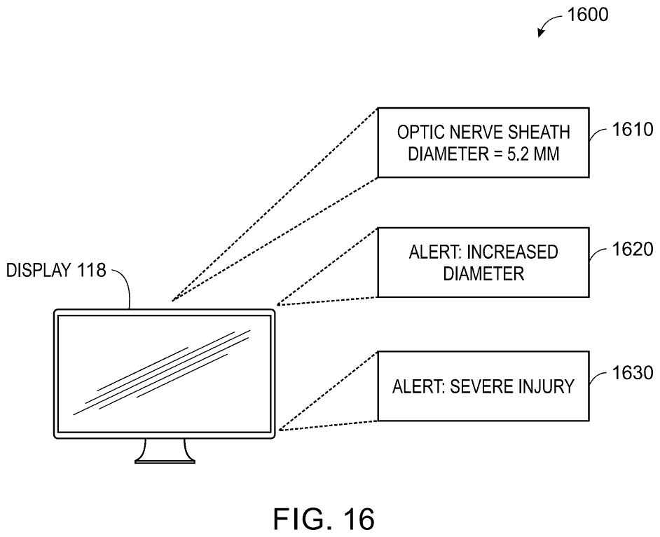

As shown in FIG. 2, the process 200 may include controlling a display to display information related to the measurement of the optic nerve sheath (operation 230). FIG. 16 is a diagram 1600 of a display of information related to a measurement of the optic nerve sheath according to an embodiment.

According to an embodiment, the information related to the measurement of the optic nerve sheath may be a value of a measurement of the optic nerve sheath. For example, the information related to the measurement may be a particular value of the measurement of the optic nerve sheath (e.g., the average value, the maximum value, etc.), a plurality of values for a plurality of measurement planes, or the like. As an example, and as shown in FIG. 16, the display 118 may display a value 1610 of the measurement of the optic nerve sheath.

According to another embodiment, the information related to the measurement of the optic nerve sheath may be an ultrasound image associated with the measurement. For example, the ultrasound image may correspond to the measurement plane in which the measurement was determined. The processor 116 may perform image processing on the ultrasound image to enhance contrast, enhance resolution, reduce speckle, or the like. An operator of the ultrasound imaging system 100 may provide a user input via the user input device 114 that selects a particular ultrasound image to be displayed. Based on the user input, the processor 116 may control the display 118 to display the particular ultrasound image. According to an embodiment, the ultrasound image may include an indicator. For example, the indicator may be an indicator of an anatomical structure, an indicator of the measurement position, or the like. Additionally, or alternatively, the ultrasound image may include the three-dimensionally segmented optic nerve sheath. An operator of the ultrasound imaging system 100 may provide a user input via the user input device 114 that modifies a segmentation of the optic nerve sheath. For example, the user input may include a point in a particular ultrasound image corresponding to a correction of the optic nerve sheath segmentation at that location. Based on the user input, the processor 116 may determine an updated measurement of the optic nerve sheath based on the modified segmentation, and control the display 118 to display the updated measurement of the optic nerve sheath.

According to another embodiment, the information related to the measurement of the optic nerve sheath may be an indicator of a change in a size of the optic nerve sheath from previous measurements. For example, the indicator of the change in the size of the optic nerve sheath may be a particular value by which the size of the optic nerve sheath has changed (e.g., +0.5 millimeters (mm), −0.1 mm, etc.). Additionally, or alternatively, the indicator of the change in the size of the optic nerve sheath may be an indicator identifying whether the size has increased, decreased, or remained substantially constant. For example, as shown in FIG. 16, the display 118 may display an indicator 1620 identifying that the size of the optic nerve sheath has increased. The processor 116 may determine the change in the size of the optic nerve sheath using a plurality of measurements of the optic nerve sheath. For example, the processor 116 may determine a change in the size of the optic nerve sheath by using the most recently determined measurements, by using a most recently determined measurement and a reference measurement, or the like. The reference measurement may be a measurement of the optic nerve sheath in which the subject was in a normal state.

According to another embodiment, the information related to the measurement of the optic nerve sheath may be a diagnosis of the subject. For example, the diagnosis may be a diagnosis of a primary injury of the subject, a secondary injury of the subject, a diagnosis of an elevated ICP level, or the like. The diagnosis may indicate a severity of the injury. For example, the diagnosis may indicate “normal,” “severe,” “mild,” or the like. As an example, and as shown in FIG. 16, the display 118 may display a diagnosis 1630 that indicates that an injury of the subject is severe. According to an embodiment, the processor 116 may input the measurement of the optic nerve sheath into the diagnosis AI model inference 128, and determine a diagnosis of the subject based on an output of the diagnosis AI model inference 128. According to another embodiment, the processor 116 may determine the diagnosis of the subject based on demographic information, vital signs, body posture, a measurement of the optic nerve sheath, and a plurality of thresholds. The thresholds may correspond to varying levels of severity of injury.

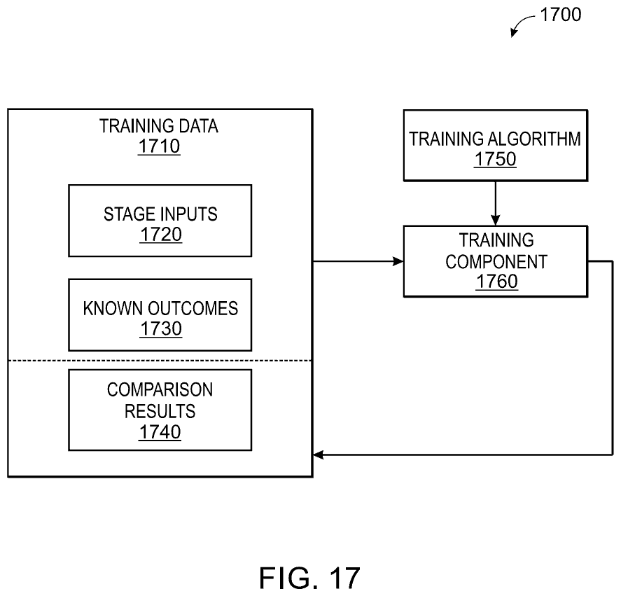

FIG. 17 is a diagram of an example process 1700 for training an AI model inference according to an embodiment. The ultrasound imaging system 100 may be configured to perform the operations of the process 1700 to train an AI model inference. The AI model inference may be the scan guidance AI model inference 122, the segmentation AI model inference 124, the measurement AI model inference 126, and/or the diagnosis AI model inference 128. According to an embodiment, the server 132 may train the AI model inference, and provide the trained AI model inference to the memory 120.

Training data 1710 may include one or more of stage inputs 1720 and known outcomes 1730 related to the AI model inference to be trained. The stage inputs 1720 may be from any applicable source including text, images, data, values, comparisons, stage outputs, or the like. The known outcomes 1730 may be included for the AI model inferences generated based on supervised or semi-supervised training. An unsupervised AI model may not be trained using known outcomes 1730. Known outcomes 1730 may include known or desired outputs for future inputs similar to or in the same category as stage inputs 1720 that do not have corresponding known outputs.

The training data 1710 and a training algorithm 1750 may be provided to a training component 1760 that may apply the training data 1710 to the training algorithm 1750 to generate the AI model inference. According to an embodiment, the training component 1760 may be provided comparison results 1740 that compare a previous output of the corresponding AI model inference to apply the previous result to re-train the AI model inference. The comparison results 1740 may be used by the training component 1760 to update the corresponding AI model inference. The training algorithm 1750 may utilize AI networks and/or models including, but not limited to, a deep learning network (e.g., Deep Neural Networks (DNNs), Convolutional Neural Networks (CNNs), Fully Convolutional Networks (FCNs), Recurrent Neural Networks (RCNs), or the like), probabilistic models (e.g., Bayesian Networks, Graphical Models, or the like), classifiers (e.g., K-Nearest Neighbors, Naïve Bayes, or the like), discriminative models (e.g., Decision Forests, maximum margin methods, or the like), models specifically discussed in the present disclosure, or the like.

An AI model inference used herein may be trained and/or used by adjusting one or more weights and/or one or more layers of the AI model inference. For example, during training, a given weight may be adjusted (e.g., increased, decreased, removed, etc.) based on training data or input data. Similarly, a layer may be updated, added, or removed based on training data/and or input data. The resulting outputs may be adjusted based on the adjusted weights and/or layers.

In light of the foregoing, some embodiments of the present disclosure provide the ultrasound imaging system 100 that improves the accuracy, efficiency, speed, and reliability of ultrasound data acquisition and optic nerve sheath measurement, and provide an improvement to the technical field of ultrasound imaging. For instance, some embodiments of the present disclosure provide the ultrasound imaging system 100 that may acquire 3D ultrasound data of an optic nerve sheath, and determine a measurement of the optic nerve sheath using the 3D ultrasound data. Further, some embodiments of the present disclosure provide the ultrasound imaging system 100 that may utilize the scan guidance AI model inference 122, the segmentation AI model inference 124, the measurement AI model inference 126, and/or the diagnosis AI model inference 128. In this way, some embodiments of the present disclosure provide the ultrasound imaging system 100 that improves the speed and efficiency of acquiring accurate ultrasound data that reflects the true anatomy of the optic nerve sheath, that reduces the sensitivity of alignment of the ultrasound probe with respect to the optic nerve sheath, that reduces the need for a skilled operator of the ultrasound probe, that improves the accuracy of optic nerve sheath measurement, and that permits optic nerves sheath measurement in a variety of time-limited and resource-limited settings.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. An ultrasound imaging system comprising:
an ultrasound probe;
a display; and
one or more processors configured to:
control the ultrasound probe to acquire three-dimensional (3D) ultrasound data of an optic nerve sheath;
determine a measurement of the optic nerve sheath based on the 3D ultrasound data; and
control the display to display information related to the measurement of the optic nerve sheath.

2. The ultrasound imaging system of claim 1, wherein the one or more processors are further configured to:
control the ultrasound probe to acquire positioning ultrasound data;
determine a scan quality metric associated with the optic nerve sheath based on the positioning ultrasound data; and
control the display to display scan guidance information that guides an operator of the ultrasound probe to position the ultrasound probe with respect to the optic nerve sheath based on the scan quality metric.

3. The ultrasound imaging system of claim 1, wherein the one or more processors are further configured to:
control the ultrasound probe to acquire positioning ultrasound data; and
determine a scan quality metric associated with the optic nerve sheath based on the positioning ultrasound data,
wherein the processor, when controlling the ultrasound probe to acquire the 3D ultrasound data of the optic nerve sheath, is configured to automatically control the ultrasound probe to acquire the 3D ultrasound data of the optic nerve sheath based on the scan quality metric.

4. The ultrasound imaging system of claim 1, wherein the one or more processors are further configured to:
control the ultrasound probe to acquire positioning ultrasound data; and
control the display to display a preview ultrasound image, that is a bi-plane ultrasound image or a multi-plane ultrasound image, in real-time with respect to the acquiring of the positioning ultrasound data.

5. The ultrasound imaging system of claim 1, wherein the one or more processors are further configured to:
control the ultrasound probe to acquire positioning ultrasound data; and
control the display to display a preview ultrasound image that is a curved plane image that follows a 3D path of an optic nerve based on the positioning ultrasound data.

6. The ultrasound imaging system of claim 1, wherein the one or more processors are further configured to:
three-dimensionally segment the optic nerve sheath based on the 3D ultrasound data; and
control the display to display the three-dimensionally segmented optic nerve sheath in a long-axis plane.

7. The ultrasound imaging system of claim 1, wherein the one or more processors are further configured to:
three-dimensionally segment the optic nerve sheath based on the 3D ultrasound data; and
control the display to display the three-dimensionally segmented optic nerve sheath in a short-axis plane.

8. The ultrasound imaging system of claim 1, wherein the information related to the measurement of the optic nerve sheath includes the measurement of the optic nerve sheath.

9. The ultrasound imaging system of claim 1, wherein the information related to the measurement of the optic nerve sheath includes an indicator in a change in a size of the optic nerve sheath.

10. The ultrasound imaging system of claim 1, wherein the information related to the measurement of the optic nerve sheath includes a diagnosis of a severity of an injury associated with the optic nerve sheath.

11. A method comprising:
controlling an ultrasound probe to acquire three-dimensional (3D) ultrasound data of an optic nerve sheath;
determining a measurement of the optic nerve sheath based on the 3D ultrasound data; and
controlling a display to display information related to the measurement of the optic nerve sheath.

12. The method of claim 11, further comprising:
controlling the ultrasound probe to acquire positioning ultrasound data;
determining a scan quality metric associated with the optic nerve sheath based on the positioning ultrasound data; and
controlling the display to display scan guidance information that guides an operator of the ultrasound probe to position the ultrasound probe with respect to the optic nerve sheath based on the scan quality metric.

13. The method of claim 11, further comprising:
controlling the ultrasound probe to acquire positioning ultrasound data; and
determining a scan quality metric associated with the optic nerve sheath based on the positioning ultrasound data,
wherein the controlling the ultrasound probe to acquire the 3D ultrasound data of the optic nerve sheath comprises automatically controlling the ultrasound probe to acquire the 3D ultrasound data of the optic nerve sheath based on the scan quality metric.

14. The method of claim 11, further comprising:
controlling the ultrasound probe to acquire positioning ultrasound data; and
controlling the display to display a preview ultrasound image, that is a bi-plane ultrasound image or a multi-plane ultrasound image, in real-time with respect to the acquiring of the positioning ultrasound data.

15. The method of claim 11, further comprising:
controlling the ultrasound probe to acquire positioning ultrasound data; and
controlling the display to display a preview ultrasound image that is a curved plane image that follows a 3D path of an optic nerve based on the positioning ultrasound data.

16. The method of claim 11, further comprising:
three-dimensionally segmenting the optic nerve sheath based on the 3D ultrasound data; and
controlling the display to display the three-dimensionally segmented optic nerve sheath in a long-axis plane.

17. The method of claim 11, further comprising:
three-dimensionally segmenting the optic nerve sheath based on the 3D ultrasound data; and
controlling the display to display the three-dimensionally segmented optic nerve sheath in a short-axis plane.

18. The method of claim 11, wherein the information related to the measurement of the optic nerve sheath includes the measurement of the optic nerve sheath.

19. The method of claim 11, wherein the information related to the measurement of the optic nerve sheath includes an indicator in a change in a size of the optic nerve sheath.

20. An ultrasound imaging system comprising:
a memory configured to store instructions; and
one or more processors configured to execute the instructions to:
control an ultrasound probe to acquire three-dimensional (3D) ultrasound data of an optic nerve sheath;
determine a measurement of the optic nerve sheath based on the 3D ultrasound data; and
control a display to display information related to the measurement of the optic nerve sheath.
